(19) United States
(12) Patent Application Publication
(10) Pub. No.: US 2006/0099183 A1
(43) Pub. Date: May 11, 2006

(54) Use of recombinant adeno-associated virus vector (rAAV) for the prevention of smooth muscle cell proliferation in a vascular graft

(76) Inventors: Pierre Zoldhelyi, Bellaire, TX (US); Janet Cunningham, Alameda, CA (US); James T. Willerson, Houston, TX (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/300,722

(22) Filed: Dec. 15, 2005

Related U.S. Application Data
(63) Continuation of application No. 10/215,164, filed on Aug. 7, 2002, now abandoned.
Application No. 10/215,164 is a continuation of application No. 09/634,729, filed on Aug. 2, 2000, now abandoned.
(60) Provisional application No. 60/146,886, filed on Aug. 2, 1999.

(51) Int. Cl. A61K 48/00

(52) U.S. Cl. 424/93.2

(57) ABSTRACT

A recombinant adeno-associated virus is used to transduce the cells of a tissue graft ex vivo. More specifically, rAAV encoding a therapeutic protein is delivered to a vascular graft to prevent smooth muscle cell proliferation or thrombosis in the graft. The cells are transfected ex vivo with the recombinant virus carrying a gene known to inhibit the proliferation and migration of vascular smooth muscle cells, thrombosis, and atherosclerosis. The methods can be used for the treatment of restenosis, vascular thrombosis, balloon injury or other vascular pathology.

This application claims priority under 35 U.S.C.§119(e) to Provisional Application No. 60/146,886, filed Aug. 2, 1999.

### FIELD OF THE INVENTION

This invention relates to a method for expressing a therapeutic protein in the cells of a tissue graft by delivering recombinant adeno-associated virus (rAAV) to such cells ex vivo. More particularly, the methods include preventing restenosis of a vascular conduit graft using rAAV carrying a gene known to inhibit thrombosis or vascular smooth muscle cell proliferation. The transduced cells or tissue is then grafted back into the damaged or occluded area.

### BACKGROUND OF THE INVENTION

Coronary artery disease, also known as atherosclerosis, is the process by which the coronary arteries become narrowed or completely occluded. Coronary artery disease can be treated by atherectomy, removal of the plaque from the inner surface of the blood vessel, coronary angioplasty using a balloon-tip catheter to open an occluded artery, or coronary bypass surgery (coronary artery bypass graft), which involves replacing diseased coronary arteries with veins or arteries obtained from the patient's lower extremities. With 400,000 coronary bypass procedures performed annually, coronary artery bypass grafting (CABG) is the most commonly performed major operation in the U.S. Saphenous vein grafts represent over 70% of grafts used, despite their suboptimal long-term patency. Thrombotic occlusion occurs within one month in 8-18% of vein grafts despite the routine use of aspirin, with 7% of vein grafts shown to be occluded by nine days. Hyperplasia, or overgrowth of cells, is the predominant mechanism of graft stenosis during the first year. One year after surgery, 15-30% of vein grafts are stenosed (up to 20% are completely occluded) and 50% of vein grafts are closed at 10 years. Accordingly, repeat procedures account for 10-20% of CABG surgery performed in the United States.

The process of coronary bypass grafting involves removing a healthy vein or artery (vascular conduit) from some part of the body, usually the femoral artery, putting that conduit into a dish with an appropriate buffer (usually a physiological saline solution such as PBS), removing the occluded or damaged artery or previously placed conduit during open heart surgery, and splicing the healthy conduit in. When reconstructing vascular tissue such as coronary arteries blocked with atherosclerosis, smooth muscle cell proliferation, thrombosis, and/or scarring frequently results in a recurrent blockage (e.g., restenosis) at the site of reconstruction.

In general, blood vessels are composed of three layers, the intima, media and adventitia. In large arteries, the intima may contain smooth muscle cells and endothelial cells. The intima is separated from the media by the internal elastic lamina. The media generally comprises smooth muscle cells and their surrounding matrix material. In large arteries, the media is comprised of defined layers of smooth muscle cells separated by elastic fibers. The adventitia forms the outermost layer of the artery wall and is separated from the media by the external elastic lamina. The adventitia is generally composed of a loose matrix containing macrophages, fibroblasts, and other cell types, as well as the vasa vasorum (a rich network of adventitial microvessels).

Restenosis of the coronary arteries occurs when vascular smooth muscle cells grow as a result of manipulation, damage, or disease, when thrombus formation occurs, and when extracellular matrix accumulates. Together, these events are responsible for the majority of occlusions of vein grafts and dilated arteries.

Gene therapy, resulting in the expression of certain therapeutic proteins in the area of graft surgery or arterial dilation could potentially prevent these vascular pathologies. Unfortunately, gene transfer into blood vessels presents several problems, including the relative inaccessibility of the vessel tissue, the inability to confine delivery to particular areas of vascular tissue, and the fact that cells of the vessel are mostly non-dividing, terminally differentiated cells. Although arterial gene transfer with marker genes or genes of biological interest has previously been reported in vivo, such methods have limited therapeutic potential due to follow-on immune responses, inefficient gene transfer, and/or the failure of the delivered gene to be expressed long-term.

Optimally, vectors used for vascular cell gene transfer should combine high efficiency with long-term expression and adequate safety. One common approach for somatic cell gene transfer, i.e., that using retroviral vectors, is not optimal for gene transfer to blood vessels because retrovirally-mediated gene transfer requires at least one cell division for integration and expression. Also, retroviral vectors and DNA liposome conjugates have a low efficiency, transducing only 0.1 to 5% of vascular endothelial and smooth muscle cells (Nabel, et al. Annu Rev Physiol 1994; 56:741-761). Similarly, other delivery methods, such as the use of “naked” DNA, are inefficient and expression is largely transient.

Adenovirus vectors have been used to transfer genes to the blood vessels, but the expression of the genes they carry is transient. Furthermore, although adenovirus vectors are designed such that they lack one or more essential viral genes (often the adenovirus E1a immediate early gene), and are thus replication deficient, they retain and express numerous viral genes in addition to the foreign gene of interest. The expression of such adenovirus proteins may be particularly problematic since many adenoviral proteins, including the fiber protein, have been shown to be cytotoxic and highly immunogenic. Hence, the production of viral proteins within target cells may present a number of health and safety concerns, perhaps the least of which includes significant local inflammation. Such inflammation can be an important factor limiting longevity of foreign gene expression and can damage healthy tissue.

Adeno-associated virus (AAV) is preferable to other gene transfer vectors because the majority of the population has been exposed to it, it is non-pathogenic, it has never been associated with any disease, and it provides for long-term gene expression. rAAV containing a gene of interest will have only AAV terminal repeat(s) necessary for replication and packaging, but no viral coding sequences which could be cytotoxic or induce an immune response to the viral translation products. Moreover, rAAV transduces many cell types without requiring active cell proliferation.

AAV is a helper-dependent DNA parvovirus that belongs to the genus Dependovirus. AAV requires co-infection with an unrelated helper virus, e.g., adenovirus, herpes virus, or vaccinia, in order for a productive infection to occur. In the absence of a helper virus, AAV establishes a latent state by inserting its genome into a host cell chromosome. Subsequent infection by a helper virus rescues the integrated viral genome, which can then replicate to produce infectious viral progeny.

AAV has a wide host range and is able to replicate in cells from any species in the presence of a suitable helper virus. For example, human AAV will replicate in canine cells co-infected with a canine adenovirus. AAV has not been associated with any human or animal disease and does not appear to alter the biological properties of the host cell upon integration. For a review of AAV, see, e.g., Berns and Bohenzky (1987) Advances in Virus Research (Academic Press, Inc.) 32:243-307.

The AAV genome is composed of a linear, single-stranded DNA molecule that contains 4681 bases (Bems and Bohenzky, supra). Strands of plus and minus polarity are both packaged, but in separate virus particles. The genome includes inverted terminal repeats (ITRs) at each end that function in cis as origins of DNA replication and as packaging signals for the virus. The ITRs are approximately 145 bp in length. The internal nonrepeated portion of the genome includes two large open reading frames, known as the AAV rep and cap regions, respectively.

The rep and cap regions code for the viral proteins that provide AAV helper functions, i.e., the proteins involved in replication and packaging of the virion. Specifically, a family of at least four viral proteins is synthesized from the AAV rep region, Rep 78, Rep 68, Rep 52 and Rep 40, named according to their apparent molecular weight. The Rep proteins are believed to be involved in viral DNA replication, trans-activation of transcription from the viral promoter, and repression of heterologous enhancers and promoters. The AAV cap region encodes at least three capsid proteins, VP1, VP2 and VP3. VP3 comprises 80% of the virion structure. For the complete sequence of the AAV-2 genome, see Vastava et al (1983) J. Virol. 45:555-64 and for a detailed description of the AAV genome, see, e.g., Muzyczka, N. (1992) Current Topics in Microbiol. and Immunol. 158:97-129.

In producing rAAV, AAV vectors can be engineered to carry a heterologous nucleotide sequence of interest (e.g., a selected gene, antisense nucleic acid molecule, ribozyme, or the like) by deleting, in whole or in part, the internal portion of the AAV genome and inserting the DNA sequence of interest between the ITRs. The ITRs remain functional in such vectors allowing replication and packaging of the rAAV containing the heterologous nucleotide sequence of interest. The heterologous nucleotide sequence is also typically linked to a promoter sequence capable of driving gene expression in the patient's target cells under the certain conditions. Termination signals, such as polyadenylation sites, can also be included in the vector.

AAV helper functions can be provided in trans via an AAV helper vector. For example, such helper vectors can be plasmids that include the AAV rep and/or cap coding regions but which lack the AAV ITRs. Accordingly, such a helper vector could neither replicate nor package itself. A number of vectors that contain the rep coding region are known, including those vectors described in U.S. Pat. No. 5,139,941, having ATCC accession numbers 53222, 53223, 53224, 53225 and 53226. Similarly, methods of obtaining vectors containing the HHV-6 homologue of AAV rep are described in Thomson et al. (1994) Virology 204:304-311. A number of vectors containing the cap coding region have also been described, including those vectors described in U.S. Pat. No. 5,139,941.

The construction of infectious recombinant AAV (rAAV) virions has been described. See, e.g., U.S. Pat. Nos. 5,173,414 and 5,139,941; International Publication Numbers WO 92/01070 (published 23 Jan. 1992) and WO 93/03769 (published 4 Mar. 1993);-Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

Contemporary rAAV virion production generally involves introduction of an AAV vector plasmid and an AAV helper plasmid into a host cell. After the AAV helper plasmid and the AAV vector plasmid bearing the heterologous nucleotide sequence of interest are introduced into the host cell (generally by stable or transient transfection), the cells are infected with a suitable helper virus in order to provide the required accessory functions. Most typically, the helper virus will be infectious adenovirus (type 2 or type 5) or herpes virus, and will, among other functions, transactivate the AAV promoters present on the helper plasmid directing the transcription and translation of AAV rep and cap regions. Upon subsequent culture of the host cells, both rAAV virions harboring the nucleotide sequence of interest and infectious helper virus particles will be produced.

Alternatively, the accessory functions can be provided on an accessory function vector without using an infectious helper virus. For example, a vector encoding the accessory functions provided by one or more adenovirus early region sequences, particularly the E1b, E2a, E4, and/or VA RNA regions, could be used in rAAV production rather than using an infectious adenovirus.

Once rAAV bearing the appropriate gene of interest is produced in a host cell line, the cells can be lysed and the rAAV can be recovered and purified using a variety of techniques. Although rAAV has previously been used to transduce a variety of tissues, efficient rAAV transduction of the vasculature resulting in long-term therapeutic gene expression to prevent restenosis after treatment of vascular pathologies has not previously been achieved.

### SUMMARY OF THE INVENTION

The methods of the present invention involve the ex vivo delivery of recombinant adeno-associated virus (rAAV) to the cells of a tissue graft in order to achieve long-term expression of a therapeutic protein in the graft. In one embodiment, rAAV encoding a protein that inhibits thrombosis, or that inhibits the proliferation of vascular smooth muscle cells, is delivered to a vascular conduit graft ex vivo. The transduced conduit is then used in a coronary artery bypass procedure to replace damaged, narrowed, or occluded coronary arteries. Accordingly, such methods can be used to prevent smooth muscle cell, endothelial cell, and fibroblast cell proliferation/accumulation, scarring, and subsequent restenosis following arterial reconstruction. In alternative embodiments, the methods can be used to treat atherogenesis, balloon injury, thrombosis, and other vascular injury.

Certain embodiments of the invention include methods for preventing smooth muscle cell, endothelial cell, and fibroblast cell proliferation/accumulation in a conduit by providing a recombinant adeno-associated virus (rAAV) containing a Gene of Interest (GOI); and transducing cells of the conduit with the rAAV ex vivo, such that the GOI expresses a protein or nucleic acid which inhibits the proliferation of the cells. In these embodiments, the GOI includes: a) those promoting the synthesis of prostacyclin, b) those stimulating cAMP formation directly, c) those inhibiting thrombosis and/or VSMC cell migration, and/or proliferation, d) those inhibiting the cell cycle-dependent kinases, e) those suppressing tumor cells by inducing apoptosis, and f) mixtures thereof. In further embodiments, the GOI is TFPI, Cox-1, E2F-1, E2F-2A, E2F-2B, PGIS, Cox-1(2A), Cox-2(2B), ENOS, TIMP-1, TIMP-2, and mixtures thereof. Alternatively, the GOI may be antisense or a ribozyme.

By transducing the graft cells ex vivo, the possibility that the immune system will react to the AAV vector itself is considerably reduced. Furthermore, because the rAAV does not encode any viral genes, no viral proteins will be present to elicit an immune response after the grafting procedure. Thus, the methods of the invention allow for efficient transduction and reduce the possibility of an inflammatory response.

### DETAILED DESCRIPTION OF THE PREFERRED EMBODIMENT

The methods of the present invention use recombinant adeno-associated virus (rAAV) to deliver a therapeutic gene of interest to the cells of graft tissue ex vivo. In certain embodiments, rAAV is used to deliver a heterologous DNA sequence encoding a therapeutic protein to the cells of a vascular conduit graft such that long-term expression of a therapeutic protein is achieved. Such methods generally involve the ex vivo transduction of the healthy conduit. More particularly, the methods may be used to transduce a healthy vein graft with rAAV encoding a therapeutic protein during coronary bypass grafting surgery. After the damaged or occluded artery is removed, the transduced vein is grafted. The subsequent expression of the rAAV-encoded protein reduces the possibility of restenosis of the vein graft by inhibiting thrombosis or vascular smooth muscle cell proliferation.

The methods disclosed herein can readily be used during surgery because the process of coronary bypass grafting generally involves removing a healthy vein or artery (conduit) from the lower part of the body and putting that conduit into a dish with an appropriate buffer while the occluded or damaged artery is being removed. Thus, the healthy conduit can be transduced with rAAV carrying the gene of interest during the surgery by placing it in a rAAV containing solution, instead of simply placing it in a physiological saline solution. After the damaged vein or artery is removed and the graft has been sufficiently incubated with rAAV, the transduced conduit can be grafted. Because the grafted vein expresses a gene that inhibits thrombosis and/or the proliferation of smooth muscle cells, restenosis at the site due to hyperplasia will be reduced or prevented.

Although AAV has previously been used to transduce tissue culture cells in vitro and vascular cells in vivo, such in vivo transduction generally occurs in an environment containing antibodies (or that is otherwise exposed to the immune system) and therefore the efficiency of viral transduction is limited. In fact, because 90% of humans have been pre-exposed to wildtype AAV2, the body and, specifically the immune system, may mount an immune response to AAV-2 antigens upon re-exposure. In addition to reducing transduction efficiency, inflammatory reactions may occur at the site of transduction, leading to tissue damage and perhaps even restenosis.

The present methods overcome this difficulty by providing efficient and safe transduction of cells of a graft with rAAV. The methods allows for the efficient transduction of the cells of the conduit in a controlled environment, where temperature, time and other conditions can be tightly regulated. This allows the technician to use less viral vector for the transduction. In addition, because the transduction is carried out ex vivo in an environment that does not contain immune cells and antibodies, the immune system cannot react to the AAV vector. Furthermore, because the rAAV does not encode viral proteins, viral translation products will not be present to elicit an immune response once the transduced graft is spliced back into the body. Accordingly, the transduction efficiency is increased and the likelihood of an inflammatory response to the virus is greatly reduced.

The practice of the present invention will employ, unless otherwise indicated, conventional methods of virology, microbiology, molecular biology and recombinant DNA techniques within the skill of the art. Such techniques are explained fully in the literature. See, e.g., Sambrook, et al. Molecular Cloning: A laboratory Manual (Current Edition); DNA Cloning: A Practical Approach, vol. I & II (D. Glover, ed.); Nucleic Acid Hybridization (B. Hames & S. Higgins, eds., current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (B. N. Fields and D. M. Knipe, eds.).

A. General Methods

The present invention provides for the successful transfer of a selected gene to a vein or arterial graft using recombinant AAV virions. The recombinant virions have been genetically altered, e.g., by the addition or insertion of a heterologous nucleic acid construct into the virus genome. The method allows for ex vivo transduction of rAAV virions into the cells of the graft, which can subsequently be introduced into a subject for treatment. A graft refers to any tissue or organ for implantation or transplantation. The invention also provides for secretion of the produced protein in vivo from transduced cells, such that restenosis caused by hyperplasia can be prevented.

Although gene transfer in vascular smooth muscle cells (VSMC'S) has previously been shown in vivo and in vitro, the combination of using rAAV as a delivery vehicle and localizing transduction to the cells of the graft is advantageous over prior techniques for a variety of reasons. First, secretion of the selected gene product will be limited to the appropriate location to produce the desired effect. Next, since the transduction is accomplished ex vivo, the transduction efficiency can be increased by controlling environmental parameters and an immune reaction to the AAV can be avoided because the transduction occurs ex vivo. Similarly, because the rAAV does not contain viral genes, viral translation products will not be present to elicit an immune response. For these and other reasons, the present methods provide significant advantages over prior gene delivery methods.

The recombinant AAV virions of the present invention, including the DNA of interest, can be produced using standard methodology, known to those of skill in the art. The methods of the present invention generally include the steps of (1) delivering rAAV to the cells of a tissue to be grafted; and (2) introducing the transduced graft to a suitable location in a patient to provide a therapeutic benefit. For example, one such method involves (1) delivering rAAV to cells of a vascular conduit graft ex vivo; (2) removing the occluded or damaged part of the coronary artery of the patient; and (3) introducing the transduced graft into the patient at the site of removal of the occluded or damaged portion. The methods of the invention contemplate that a variety of tissues may be transduced with rAAV ex vivo, and that the rAAV can encode one of several genes, that when expressed, will provide a therapeutic benefit to the patient.

1. AAV Vectors

The AAV vectors of Podsakoff, et al. U.S. Pat. No. 5,858,351, issued Jan. 12, 1999 can be used for the present invention (herein incorporated by reference). In summary, AAV vectors are generally constructed to provide, as operatively linked components, elements which direct transcription, control elements including a transcriptional initiation region, a DNA sequence of interest and a transcriptional termination region. The control elements are selected to be functional in a mammalian cell. The resulting construct containing the operatively linked components is joined to one or more functional AAV ITR sequences.

The nucleotide sequences of AAV ITR regions are known. The AAV ITRs used in the present invention may be derived from any of several AAV serotypes, including without limitation, AAV-1, AAV-2, AAV-3, AAV-4, AAV-5, AAVX7, etc. Furthermore, where 5′ and 3′ ITRs flanking a selected nucleotide sequence in an AAV expression vector are used, they need not necessarily be identical or derived from the same AAV serotype or isolate so long as they function as intended.

A number of heterologous genes may be used to inhibit restenosis. The choice of heterologous gene is detailed below, but can be any gene that inhibits smooth muscle cell proliferation, or otherwise inhibits restenosis, including TFPI and E2F. A heterologous nucleic acid refers to a nucleic acid sequence placed outside of the region of nucleic acid in which it occurs in nature.

The selected nucleotide sequence, such as TFPI or another gene of interest, is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter; mouse mammary tumor virus LTR promoter; adenovirus major late promoter (Ad MLP); a herpes simplex virus (HSV) promoter; a cytomegalovirus (CMV) promoter such as the CMV immediate early promoter region (CMVIE); a Rous sarcoma virus (RSV) promoter; synthetic promoters; hybrid promoters; and the like. In addition, sequences derived from nonviral genes, such as the murine metallothionein gene, will also find use herein. Such promoter sequences are commercially available from, e.g., Stratagene (San Diego, Calif.).

For purposes of the present invention, control elements, such as muscle-specific and inducible promoters, enhancers and the like, will be of particular use. Such control elements include, but are not limited to, those derived from the actin and myosin gene families, such as from the myoD gene family; the myocyte-specific enhancer binding factor MEF-2, control elements derived from the human skeletal actin gene and the cardiac actin gene; muscle creatine kinase sequence elements, and the murine creatine kinase enhancer (mCK) element; control elements derived from the skeletal fast-twitch troponin C gene, the slow-twitch cardiac troponin C gene and the slow-twitch tropinin I gene; hypoxia-inducible nuclear factors, steroid-inducible elements and promoters, such as the glucocorticoid response element (GRE); the fusion consensus element for RU486 induction; and elements that provide for tetracycline regulated gene expression.

The AAV vector harboring the gene of interest can be constructed by inserting a heterologous DNA sequence into an AAV genome that has had the major AAV open reading frames (ORFs) excised therefrom. Other portions of the AAV genome can also be deleted, so long as a sufficient portion of at least one ITR remains to allow for replication and packaging functions.

2. Choice of Heterologous DNA Sequences

Suitable DNA molecules for insertion in rAAV will generally be less than about 5 kilobases (kb) in size and will include, for example, a gene that encodes a protein that prevents smooth muscle cell proliferation and/or migration and/or thrombosis and/or monocyte accumulation and atherogenesis in a vein or artery.

Suitable DNA molecules include, but are not limited to, those encoding proteins which: 1) promote the synthesis of prostacyclin (thereby enhancing cAMP synthesis in receptor-mediated fashion), such as COX-1, COX-2, and PGIS; 2) stimulate cAMP formation more directly (the Gs alpha subunit and adenylyl cyclase; 3) inhibit thrombosis and/or vascular smooth muscle cell migration and/or proliferation, such as TFPI, eNOS, TIMP-1, and TIMP-2; 4) inhibit the cell cycle-dependent kinases, such as p21; 5) suppress tumor cells by inducing apoptosis, such genes such as E2F-1; and 6) mixtures thereof. Examples of the involvement of some of the factors in restenosis are detailed below.

Alternatively, the DNA molecule could be an antisense or other molecules such as ribozymes which inhibit the expression of genes known to be involved in proliferation/migration of VSMC, or those involved in thrombosis or any part of restenosis.

TFPI is an inhibitor of tissue factor. Tissue factor (TF) is the cellular initiator of thrombin generation and blood coagulation in hemostasis and thrombosis. After its exposure in the injured vessel wall, TF binds to circulating factor VIIa, which in the extrinsic pathway of blood coagulation activates factor X. Factor Xa converts prothrombin to thrombin, which activates platelets, converts fibrinogen to fibrin, and exerts positive feedback on its generation through platelet activation and activation of factors V, VIII and XI. TF-driven thrombin generation plays a pivotal role in arterial and venous thrombosis and can activate enzymes involved in neointima formation, including matrix metalloproteinases. TF itself promotes migration of vascular smooth muscle cells (VSMC), whereas factor Xa and thrombin are potent mitogens for VSMC. TF is released and upregulated after vascular injury and initiates a cascade of events that may play a role in restenosis after percutaneous revascularization interventions. TF in the atherosclerotic wall appears to contribute importantly to the thrombogenicity of the disrupted atherosclerotic plaque and its accumulation has been observed in experimental vein grafts and arteries, where it is believed to be involved in chronic thromboproliferative graft deterioration and post-injury restenosis.

In addition to wild-type TFPI, variant TFPI sequences can be used in order to enhance the intrinsic antithrombotic and potential anti-restenosis effects of TFPI. For example, hybrid/chimeric proteins and C-terminus truncated TFPI may be used, particularly, as C-terminally truncated molecules have been shown in endpoint assays to inhibit factor Xa to the same extent as full-length TFPI.

The transcription factor, E2F-1 promotes S-phase entry and cell death in transformed cells and cardiomyocytes. Recently, it was shown that growth of early (less than or equal to 5) passage human coronary vascular smooth muscle cells is regulated by E2F-1. Therefore, E2F-1 can be used therapeutically to regulate growth and cellularity of human vascular smooth muscle and endothelial cells by forcing them to enter S-phase and then to die.

Prostacyclin (or PGI₂) can be delivered therapeutically according to the present invention to inhibit platelet secretion and aggregation, relieve vascular contraction, and/or suppress smooth muscle cell proliferation and lipid accumulation in vascular smooth muscle and mononuclear cells. PGI₂ is a potent platelet inhibitor and vasorelaxant molecule that is synthesized by the healthy endothelium. PGI₂ is released by the healthy endothelium and acts in a paracrine manner on platelets on the luminal side and smooth muscle cells underneath the endothelium. In aqueous solution, it has a short half-life of approximately 3 minutes. Its actions involve receptor-mediated activation of adenylate cyclase, leading to increased intracellular cAMP levels.

COX-1 is involved in the key rate-limiting step in prostacyclin biosynthesis. Consequently, delivery of COX imparts a vasoprotective effect and may further confer antithrombotic protection.

3. Production of rAAV Virions

To produce rAAV virions, the recombinant AAV vector containing a heterologous DNA sequence is introduced into a suitable host cell using known techniques, e.g., by transfection. For the purposes of the invention, suitable host cells for producing rAAV virions include microorganisms, yeast cells, insect cells, and mammalian cells, that can be, or have been, used as recipients of a heterologous DNA molecule.

In order to replicate and encapsidate the sequence of the AAV vector, host cells containing such AAV vectors must also be rendered capable of providing the AAV helper functions that were deleted from the AAV vector, namely the rep and cap coding regions, or functional homologues thereof. Such AAV helper functions can be provided in trans by transfecting the host cell with an AAV helper vector either prior to, or concurrently with, the transfection of the AAV vector containing a heterologous DNA molecule. AAV helper vectors lack the AAV ITRs and thus can neither replicate nor package themselves. Both AAV vectors and AAV helper vectors can be constructed to contain one or more optional selectable markers.

As mentioned, AAV production generally requires coinfection with an unrelated helper virus (e.g., an adenovirus, a herpes virus or a vaccinia virus) in order to supply necessary “accessory functions.” For example, it has been demonstrated that adenovirus supplies factors required for AAV promoter expression, AAV messenger RNA stability and AAV translation. See, e.g., Muzyczka, N. (1992) Curr. Topics. Microbiol. and Immun. 158:97-129. In the absence of these “accessory functions,” AAV establishes a latent state by insertion of its genome into a host cell chromosome. On the other hand, upon supply of these accessory functions, the integrated copy is rescued and can replicate to produce infectious viral progeny.

Rather than using infectious helper virus to provide the necessary accessory functions, the present invention contemplates that the accessory functions will be provided on a replication-incompetent accessory function vector. In certain embodiments, the accessory function vector(s) will include adenoviral-derived nucleotide sequences necessary for rAAV virion production. Such sequences can include E1a, E1b, E2a, E4 and VA RNA regions.

More specifically, U.S. Pat. No. 6,004,797, incorporated by reference herein, describes the production of rAAV without infectious helper virus. For example, instead of infecting with infectious adenovirus, the host cell is transfected with one or more vectors having nucleotide sequences from an adenovirus type-2 or type-5 genome that are required for rAAV replication and packaging but are insufficient to make infectious adenovirus. In certain embodiments, these nucleotide sequences include (i) adenovirus VA RNAs, (ii) an adenovirus E4 ORF6 coding region, (iii) an adenovirus E2a 72 kD (coding for the E2a 72 kD DNA-binding protein), or any combination of nucleotide sequences (i), (ii), and (iii). Importantly, these accessory function vectors lack most of the adenovirus genome, including the fiber protein. Production of rAAV in this manner, i.e., without infectious helper virus, minimizes the health and safety concerns.

The accessory function vectors of the invention can alternatively include one or more polynucleotide homologue(s) that replace the adenoviral gene sequences, so long as each homologue retains the ability to provide the accessory functions of the replaced adenoviral gene. Thus, homologous nucleotide sequences can be derived from another adenoviral serotype (e.g., adenovirus type-2), from another helper virus moiety, or can be derived from any other suitable source.

In particular, members of herpesviridae are known to act as AAV helper viruses. Thus, in alternative embodiments of the disclosed invention, particular sequences from herpesviridae capable of assisting in the production of AAV in a host cell can be used in an accessory function vector.

Further, accessory function vectors constructed according to the invention can be in the form of a plasmid, phage, transposon or cosmid. Alternatively, the vector can be in the form of one or more linearized DNA or RNA fragments that, when associated with the appropriate control elements and enzymes, can be transcribed or expresses in a host cell to provide accessory functions.

Accessory functions can be engineered using conventional recombinant techniques. Particularly, nucleic acid molecules can be readily assembled in any desired order by inserting one or more accessory function nucleotide sequences into a construct, such as by ligating restriction fragments into a cloning vector using polylinker oligonucleotides and the like. The newly formed nucleic acid molecule can then be excised from the vector and placed in an appropriate expression construct using restriction enzymes or other techniques that are well known in the art. The resulting accessory function vector can then be transfected into the host cell line prior to, concurrently with, or after the introduction of the AAV vector containing the heterologous DNA sequence and the AAV helper vector.

In alternative embodiments, rAAV may be produced using various cell lines that possess AAV helper and/or accessory functions necessary to produce rAAV particles, thus obviating the need for transduction with helper and/or accessory function vectors.

Following recombinant AAV production, the host cell line can be lysed and the rAAV virions can be purified using a variety of conventional purification methods, such as a variety of column purification techniques or CsCl gradients. The resulting rAAV virions can then be used to deliver the heterologous DNA to suitable tissue graft cells.

4. Transduction of the Vascular Conduit

Generally, rAAV virions are introduced into the cells of a vascular conduit (artery or vein) ex vivo. A vascular conduit denotes an artery, a vein, or an artificial conduit.

The conduits are meant to include those that are connected entirely (i.e., both ends) into an artery (i.e., a vein graft); those where only one end of the vein (conduit) is connected with the artery and the other end is left in situ (an arteriovenous fistula); or those that are made of artificial material, such as a “stent-graft”, and are seeded with cells before insertion of the stent-graft into an artery.

The saphenous veins are most commonly used for the coronary bypass techniques. The saphenous veins are two veins, the great and the small, which serve as the principle veins running superficially up the leg.

Appropriate doses of rAAV will depend on, among other factors, the mammalian type (e.g., human or nonhuman primate or other mammal), age, size and general condition of the graft to be treated, as well as the promoter used, the transduction efficiency, the particle infectivity, and the particular therapeutic protein that is being expressed. An appropriate therapeutically effective amount can be determined by one of skill in the art through clinical trials, but will be an amount sufficient to confer a therapeutic benefit upon expression of protein encoded by the heterologous gene delivered. For example, in treating vascular conduits for use in coronary bypass surgery, the amount of rAAV delivered will be that sufficient to inhibit vascular smooth muscle proliferation and/or thrombosis.

5. Test to measure flow of blood in the artery

Blood flow velocities were tested by doppler flow probe analysis.

B. Experimental

TFPI is believed to suppress the development of chronic vascular stenosis and vein graft deterioration by interrupting tissue factor (TF) and thrombin-dependent mechanisms of recurrent thrombosis and intimal hyperplasia. Briefly, tissue factor (TF) is the cellular initiator of thrombin generation and blood coagulation. TF-driven thrombin generation results in fibrinogen clotting and platelet activation and the release of mitogens to the injured vessel wall. In addition, TF promotes vascular smooth muscle cell (VSMC) migration, whereas factor Xa and thrombin are VSMC mitogens. Tissue factor pathway inhibitor (TFPI) is an endogenous inhibitor of TF and factor Xa, and may inhibit thrombosis and neointima formation after vascular injury.

In more detail, tissue factor (TF), a transmembrane protein receptor, is the cellular initiator of thrombin generation and blood coagulation in hemostasis and thrombosis. After its exposure in the injured vessel wall, TF binds to circulating factor VIIa, which in the extrinsic pathway of blood coagulation activates factor X. In addition, the TF/factor VIIa complex activates factor IX, leading to the generation of factor Xa in a second (intrinsic) pathway. Within the membrane-bound prothrombinase complex, factor Xa converts prothrombin to thrombin, which activates platelets, converts fibrinogen to fibrin, and exerts positive feedback on its generation through platelet activation and activation of factors V, VIII and XI. TF-driven thrombin generation plays a pivotal role in arterial and venous thrombosis and can activate enzymes involved in neointima formation, including matrix metalloproteinases. TF itself promotes migration of vascular smooth muscle cells (VSMC), whereas factor Xa and thrombin are potent mitogens for VSMC. In addition, platelets, activated during thrombogenesis, release mitogens and chemotaxins to VSMC in the injured vessel wall and the extent of platelet-thrombus deposition to the injured vessel wall closely correlates with the severity of the fibroproliferative response. Thus, TF, which is released and upregulated after vascular injury, initiates a cascade of events that are likely to play a major role in restenosis. Moreover, the increased presence of TF in the atherosclerotic wall appears to contribute importantly to the intense thrombogenicity of the disrupted atherosclerotic plaque and TF accumulation has been observed in a vein graft model, where it may play a role in chronic graft deterioration.

Recently, administration of tissue factor pathway inhibitor (TFPI), the endogenous inhibitor of TF, has been studied as a new approach to the prevention of thrombin generation, thrombosis, and restenosis after PRI in animal models of arterial injury. At physiological concentrations, TFPI forms initially a complex with factor Xa, and this complex associates with, and inhibits, TF/factor VIIa, whereas higher concentrations of TFPI can inhibit tissue factor/factor VIIa in the absence of factor Xa(2). Thus, TFPI functions to exert negative feedback on TF-dependent thrombin generation in a factor XA (prothrombinase)-dependent fashion and may have advantages over the specific antithrombins, which have little influence on the ongoing thrombin generation in stable and unstable coronary disease.

Long-term, rather then short-lived administration of TFPI will likely be required for inhibition of restenosis. Unless its administration can be extended beyond the first days after the vascular insult, it will only produce a temporary effect. For instance, whereas a short-term administration of the specific antithrombin, hirudin at the time of balloon injury was shown to have little or no effect on the development of chronic stenosis in animal models of arterial injury and in patients after percutaneous revascularization interventions (PRI), a continuous 14-day infusion of hirudin or local hirudin gene transfer reduced neointimal formation in arterial injury models. This underscores (a) the pivotal role of thrombin and of thrombus-derived mitogenic, chemotactic, and prothrombotic factors in the pathogenesis of the arterial restenosis and (b) the requirement for prolonged thrombin inhibition to achieve long-term effects.

While continuous systemic administration of antithrombotic agents, including hirudin, TFPI, or anti-platelet agents, may promote long-term patency of injured vessels, some hemorrhagic risks are inevitable. Moreover, the systemic doses of recombinant human TFPI capable of preventing arterial thrombosis and, potentially, restenosis are substantial (100 μg/kg.min), raising further questions on the practicality of a systemic dosing approach. Indeed, the need for such high dosage has substantially tempered the development of TFPI as an antithrombotic agent for the treatment of arterial disorders.

In contrast to pharmacological approaches to thrombotic and thromboproliferative disorders, local gene transfer techniques restrict the presence of a potentially therapeutic protein to the vascular site at risk, while extending in time the effect of a single administration of the therapeutic precursor, the DNA encoding a potentially beneficial protein. In addition, the effect of locally expressed proteins is often greater than could be predicted from systemic dosing schedules. The relative inefficacy of circulating TFPI vis-à-vis arterial thrombosis is also supported by the observation that plasma levels of endogenous TFPI are markedly elevated in patients with unrelieved acute coronary thrombosis and that the presence of endogenous TFPI in the low pg/mg range in carotid endarterectomy specimens was shown to attenuate local TF activity measured ex vivo in these specimens. Therefore, by expressing the protein locally, the relatively large doses required for systemically administered recombinant TFPI (in the mg range) will not be necessary (Willerson JT, Unpublished Observations, 1995). Together, these observations emphasize the potent effects and relatively greater safety of TFPI expressed in situ compared to its systemic administration.

Below are examples of specific embodiments for carrying out the methods of present invention, i.e., methods for conferring long-term expression of a therapeutic protein in tissue grafts through the ex vivo delivery of rAAV. The examples are offered for illustrative purposes only, and are not intended to limit the scope of the present invention in any way.

### EXAMPLE 1

### Use of AAV to Transduce a Rabbit Jugular Vein ex vivo—expression of LacZ

Expression of LacZ was demonstrated 2 weeks after placing a rAAV-LacZ transduced venous segment into the carotid artery of rabbits. The rAAV-LacZ used in this example was prepared generally as described in U.S. Pat. No. 5,858,351, incorporated by reference herein.

The method was performed as follows:

Three New Zealand White rabbits, 12-16 months of age were anesthetized with ketamine and xylazine, intubated and ventilated. The anterior aspect of the neck was shaved and prepared in a sterile manner. EKG, heart rate, and transcutaneous oxygenous saturation were monitored intraoperatively. Through a midline incision in the ventral area of the neck, the right jugular vein was gently exposed. After administration of heparin IV (100 units/kg) both the external and internal jugular vein was clamped. A small canula was introduced in the external jugular vein and secured by suture. The vein was gently irrigated with PBS solution. Thereafter, the isolated vein graft was infused with an appropriate amount of viral solution ex vivo (containing rAAV at a concentration of about 1-6×10¹¹ particles/mL) and allowed to incubate at room temperature for 30 minutes. In the meantime, the right carotid artery was carefully exposed and clamped at its proximal and distal end.

After incubating the vein, the rAAV was removed, the vein was rinsed with PBS, and the vein was interposed in reversed fashion and end-to-side into the ipsilateral carotid artery (Prolene 8-0, Ethicon). Blood flow was re-established by release of the arterial clamps. Hence, the carotid artery was divided so blood would flow through the grafted vein and none through the carotid artery at the level of the graft. Doppler evaluation was performed to record baseline blood flow proximal and distally to the graft and at the level of the graft itself. The neck wound was closed in layers and the skin was closed by single staples. A single dose of antibiotic (cefonicid sodium, 35-40 mg) was administered intraoperatively. To avoid any postoperative pain, buprenorphine (0.02-0.05 mg/kg) was administered subcutaneously. After extubation, the rabbits were allowed to return to their housing.

On the day after surgery, all animals were placed on a 1% cholesterol diet until sacrifice to promote graft atherosclerosis. At the time of sacrifice, the venous graft was exposed first. After clinical examination, doppler evaluation was performed to record blood proximally and distally to the graft and at the level of the graft. Then, the contralateral side of the neck was exposed to obtain carotid doppler flow measurements. Rabbits were euthanized with Beuthanasia-D (2 ml IV). The vein graft (with adjacent carotid artery) and contralateral jugular vein and carotid artery are pressure perfused at 100 mm Hg with 10% buffered formaldehyde and harvested into 10% formaldehyde solution for histological evaluation only. X-Gal staining was performed in standard fashion and revealed β-galactosidase activity 14 days after the vein was grafted.

The finding of vein graft transduction by rAAV ex vivo, and prior to grafting the vein was also confirmed in two more rabbits.

This example demonstrates that rAAV can deliver a heterologous gene to venous graft tissue ex vivo. It further demonstrates that the protein encoded by the heterologous gene can be expressed in such graft tissue, and that that expression can be maintained for at least 14 days after the grafting procedure.

### EXAMPLE 2

### Use of AAV to Transduce a Rabbit Jugular Vein ex vivo—Expression of TFPI

The expression of therapeutic levels TFPI is achieved in a mammalian vein graft by ex vivo delivery of rAAV to the graft tissue. Preparation of rAAV-TFPI is accomplished using techniques known in the art and generally outlined previously.

The jugular vein from Male Watanabe rabbits is removed and transduced with rAAV. rAA V-CMV-TFPI, and rAAV-CMV-null (no foreign gene) are delivered to the removed vein in a solution having a concentration of about 5×10¹⁰ to about 6×10¹¹ particles/mL rAAV. Transduction is allowed to proceed for about 30 min. The jugular vein is then grafted into the animal. Animals are sacrificed 7, 14, 30, and 90 days after surgery, and tissues are embedded and frozen in TBS to perform TFPI immunostaining. Twelve rabbits will be used per group.

These studies will give information on the efficacy of TFPI gene transfer to prevent neointima formation in a rabbit model of mildly/moderately severe atherosclerosis. The ability of locally overexpressed TFPI to inhibit TF activity is assessed.

The rabbit TFPI gene will be isolated as follows: The total RNA from New Zealand rabbit lung tissue is isolated by the guanidinium thiocyanate-acid phenol method. First strand cDNA is generated with 1 mg of total RNA primed with oligo dT using SuperscriptII (GIBCO) according to manufacturer's protocol. The cDNA pool is then diluted 1:20 and used as template for PCR. PCR primer pairs employed were rTFPI.ATG.forward (ggggtaccatgggaaagaaagaacacatcttttgg) (SEQ ID NO:1) and rTFPI.TAA.reverse (gctctagattatgtctttttaacaaaagtttctac) (SEQ ID NO:2) corresponding to the N— and C-terminal regions of rabbit TFPI cDNA. The forward primer has Kpnl and Ncol sites with a Kozak sequence. The reverse primer has an Xbal site with stop codon. The restriction sites are underlined and encoding sequences are in bold. Amplification is carried out with the following PCR conditions using pfu DNA polymerase: 5 min at 94° C. and 3 min at 65° C. to generate second strand cDNA; then 30 sec at 94° C., 30 sec at 53° C., and 90 sec at 72° C. for 30 cycles; and a final extension for 5 min at 72° C. The amplified cDNA was cloned into pACCMV-pLpA and sequenced.

In the jugular vein interposition graft in cholesterol-fed rabbits, the vein is transduced ex vivo using nonviral or viral gene transfer, and grafted in reversed fashion to the ipsilateral carotid artery. In one study, endothelial cells in the vein grafts infected with recombinant adenovirus at a titer of 1×10⁹ pfu/mL expressed the E. Coli LacZ gene for 7 days. Neoexpression of VCAM-1 and ICAM-1 and infiltration with polymorphonuclear cells occurred to a similar degree in both virus and nonvirus (control) treated veins.

The jugular vein will be harvested and incubated for 30 min with four different doses of rAAV-CMV-LacZ: 1×10¹⁰, 5×10¹⁰, 1×10¹¹, 6×10¹¹ particles/mL and rAAV-CMV-TFPI. Although a dose of 6×10¹¹ particles/mL appears necessary in preliminary experiments, it is possible that a lower titer will be required. In order to gain information with this new approach (rAAV-mediated gene transfer to vein grafts), animals are harvested 7, 13, 28 days and 3 months after surgery and evaluated with routine and X-gal histochemistry for gene expression and inflammation.

On the day after surgery, New-Zealand-White rabbits are placed on a 1% cholesterol diet and kept on this diet until sacrifice. Vein grafts are incubated for 30 min in rAAV. Animals are randomized to receive optimal titer of rAAV-CMV-TFPI or rAAV-CMV-null (maximum expression without inflammation), as established in the rAAV-CMV-LacZ dose-finding studies. The rabbits are sacrificed after 2 weeks and after 1 month and 3 months. Doppler evaluation is performed to record baseline blood flow proximal and distally to the graft and at the level of the graft itself. Because these animals will serve both to assess gene expression of TFPI and for histomorphometric evaluation of lesion formation, the grafted arteries are embedded at sacrifice in cryoprotective compound (TBS), with great care to preserve the morphology of the vein. At sacrifice, blood flow is evaluated by doppler flow probe in the grafted and contralateral artery. If there is prevention of lesion formation at 3 months, the observation will be extended up to 6 months. 12 completed rabbits are used per group.

TFPI gene expression is assessed by immunohistochemistry or ELISA using a TFPI antibody and following the manufacturer's directions. Additionally, at least 24 hours after grafting of the conduit, the conduit will be harvested and kept in culture for 4 days. Human TFPI will be assessed in the culture medium.

TFPI immunohistochemistry-Animals will be sacrificed without administration of heparin to prevent potential release of TFPI from endothelial cells. Tissue will be embedded in cryoprotective compound (TBS). Five-μm tissue sections will be fixed for 15 minutes in 4% formaldehyde in PBS and exposed for 10 minutes to 3% H₂O₂. The sections will be blocked in 2% horse serum and exposed for 1 hour at room temperature to a monoclonal antibody to human TFPI (American Diagnostica) or cytomegalovirus (DAKO, Carpinteria, Calif.) followed by incubation in a biotinylated horse antimouse antibody (Vector, Burlingame, Calif.) and streptavidin-biotin-horse radish peroxidase (Vector). Antibody binding will be visualized by exposure to DAB and sections are counterstained in 1% Alcian blue/2% methyl green or hematoxylin-eosin. PBS is used for all washes.

The presence of inflammation and TF immunoreactivity are also assessed by immunohistochemistry (as explained for TFPI) as well as by blood coagulation (platelet aggregation) and bleeding times. Coagulation variables and bleeding times are measured by the following protocol: Blood samples are drawn from a femoral vein through an 18 gauge untreated polyethylene catheter and collected into 3.8% sodium citrate (1:9=vol/vol) or sodium heparin (2 units/ml). Coagulation variables including activated coagulation times (ACT), prothrombin time (PT), and partial thromboplastin times (aPTT) are measured in a Hemochron 80 dual coagulation system according to the manufacturer's instructions (International Technidyne, Madison, N.J.). Ear skin bleeding times (BT) are measured before grafting (control, day 0) and on the day of flow measurement (day 5), using a Simplex II device (Organon Teknika, Durham, N.C.).

The extent of atherosclerotic neointima formation is assessed by carotid blood flow velocity using the Doppler flow assay.

Preliminary experiments have shown that it is possible to mutate TFPI to produce a more potent inhibitory molecule. Therefore, Example 3 will be performed using the following mutants:

### EXAMPLE 3

### Use of AAV to Transduce a Rabbit Jugular Vein ex vivo—Expression of TFPI Mutants\Hybrids

Mutants/hybrids of TFPI are generated and delivered to graft tissue using rAAV according to the techniques described in the previous examples and in the Detailed Description. The delivery of such molecules provides a therapeutic benefit by inhibiting, in addition to TF and factor Xa, integrins involved in platelet aggregation and smooth muscle cell migration. Such molecules may also provide therapeutic benefit by anchoring TFPI to the surface vascular smooth muscle cells exposed by mechanical injury. TFPI variants that enhance the intrinsic antithrombotic and potential anti-restenosis effects of TFPI are also used.

Alternatively, truncated forms of TFPI, e.g., one lacking the third Kunitz-type domain and carboxy-terminus (TFPI₁₋₁₆₁) is delivered to graft tissue using rAAV. It will be appreciated by one of skill in the art that a number of TFPI variants and hybrids/chimeras may be identified and used in a similar manner to provide therapeutic benefit.

### EXAMPLE 4

### Use of AAV to Transduce a Human Saphenous Vein ex vivo—Expression of TFPI and TFPI Variants

rAAV encoding TFPI and TFPI variants is used to transduce a human saphenous vein ex vivo. A typical coronary bypass surgery is performed as follows: There are 3 main coronary arteries from the base of the aorta, a right and a left which branches into 2 arteries that feed the heart muscle. When they are blocked with atherosclerosis, either angioplasty is performed or coronary bypass surgery is performed. Coronary bypass surgery begins with an incision down the center of the chest. The surgeon opens the chest to expose the heart and opens the pericardium to expose the vessels. A venous graft is procured from the leg (saphenous vein) or artery (internal mammary artery). EKG, heart rate and transcutaneous oxygenous saturation are monitored intraoperatively. The vein is placed into a sterile tissue culture dish, is infused with a solution containing an appropriate amount of rAAV, and is allowed to incubate at room temperature for a time sufficient for the rAAV to transduce the cells of the graft. In the meantime, the atherosclerosed part of the coronary artery is carefully removed. Usually one surgery team is removing the saphenous vein while the other removes the occluded artery. After the incubation period, rAAV is removed, the vein is rinsed with a buffer, and the vein is grafted into the area where the atherosclerotic part of the artery was removed. More specifically, one end of the vein is attached to the aorta and the other end is attached distal to the blockage. Blood flow is re-established by release of the arterial clamps. Hence, the carotid artery was divided so blood will flow through the grafted vein and not through the carotid artery at the level of the graft. Doppler evaluation is performed to record baseline blood flow proximal and distally to the graft and at the level of the graft itself. The neck wound is closed in layers and the skin is closed. Antibiotic (cefonicid sodium, 35-40 mg) is administered intraoperatively.

A method to transduce other heterologous genes with recombinant rAAV vectors into conduits ex vivo and the use of this method to obtain in vivo expression of that gene after insertion (grafting) of the rAAV-transduced conduit into a natural vascular bed is set out in Example 5.

### EXAMPLE 5

### Use of AAV to Transduce a Human Saphenous Vein ex vivo for the Treatment of Restenosis—E2F-1 and Other Heterologous Genes

rAAV encoding E2F-1 is used to transduce a human saphenous vein ex vivo. However, the TFPI gene is removed and replaced with a heterologous gene less than about 5 kilobases (kb) in size and will include, for example, a gene that encodes a protein that prevents smooth muscle cell proliferation in a vein or artery.

Other suitable DNA molecules for use in AAV vectors will be less than about 5 kilobases (kb) in size and will include, for example a gene that encodes a protein that prevents smooth muscle cell proliferation and/or migration and/or thrombosis and/or monocyte accumulation and atherogenesis in a vein or artery. Suitable DNA molecules include, but are not limited to, those encoding proteins 1) promoting the synthesis of prostacyclin (thereby enhancing cAMP synthesis in receptor-mediated fashion), such as COX-1, COX-2, and PGIS; 2) stimulating cAMP formation more directly (the Gs alpha subunit and adenylyl cyclase; 3) inhibiting thrombosis and/or vascular smooth muscle cell migration and/or proliferation, such as TFPI, eNOS, TIMP-1, and TIMP-2; 4) inhibiting the cell cycle-dependent kinases, such as p21; 5) suppressing tumor cells by inducing apoptosis, such genes such as E2F-1; and 6) mixtures thereof Examples of such proteins include PGI2, TFPI, Cox-1, E2F-1, E2F-2A, E2F-2B, PGIS, Cox-1(2A), Cox-2(2B), ENOS, TIMP-1, and TIMP-2.

The saphenous vein is removed from the patient. EKG, heart rate and transcutaneous oxygenous saturation are monitored intraoperatively. The vein is placed into a sterile tissue culture dish and transfected. Thereafter, the viral solution is infused into the vein ex vivo at the doses indicated and allowed to incubate at room temperature for 30 min in the segment. In the meantime, the heart is arrested with cardioplegia solution, the aorta is cross-clamped, and the patient is placed on extracorporeal perfusion. The atherosclerotic, obstructed part of the coronary artery or previous vein graft is carefully removed. After the incubation period, rAAV is removed, the vein is rinsed with PBS, and the vein is grafted as an aorto-coronary saphenous vein graft into the area where the atherosclerotic part of the artery was removed. Blood flow is re-established by release of aortic cross clamping and initiation of spontaneous cardiac contractions, and termination of extracorporeal perfusion.

### EXAMPLE 6

### Use of the Method in Transplantation of Organs and Reattachment of Severed Limbs

The method disclosed herein can be used for any type of surgery which potentially damages conduits and leads to restenosis. For example, transplantation surgeries often involve the reattachment of major veins and arteries. Before such conduits are reattached, the AAV vector which is capable of expressing a restenosis inhibitory protein is transfected into the vein or artery as described in Example 3. The transfection is allowed to proceed for up to 30 minutes and the vein or artery is attached during transplantation surgery. Alternatively, the method can be used during reattachment of a limb or body part. Such body parts contain veins or arteries which it is necessary to attach. Therefore, the AAV virus vector containing the restenosis inhibiting protein is transfected into the vein or artery of the organ for up to 30 minutes. Then the body part is attached and the vein or artery is attached. Examples of such uses include but are not limited to: liver, pancreas, and kidney transplantation, pharyngeal reconstruction, vein interposition grafts for stroke or phlebitis, and in the prevention of graft vasculopathy.

What is claimed is:

1. A method of administering a recombinant adeno-associated virus (rAAV) virion to a mammalian vascular conduit, comprising:
contacting said rAAV virion with said mammalian vascular conduit ex vivo, wherein said contacting results in transduction of said mammalian vascular conduit by said rAAV virion.

2. The method of claim 1, wherein the vascular conduit is an artery.

3. The method of claim 1, wherein the vascular conduit is a vein.

4. The method of claim 1, wherein the vascular conduit is an artificial conduit.

5. The method of claim 1, wherein the method further comprises grafting the transduced vascular conduit into a mammalian subject.

6. The method of claim 1, wherein said rAAV virion comprises a heterologous nucleic acid.

7. The method of claim 6, wherein said heterologous nucleic acid is a heterologous gene.

8. The method of claim 1, wherein said rAAV virion comprises a gene of interest.

9. The method of claim 8, further comprising expressing the gene of interest in the vascular conduit, thereby resulting in an expression product.

10. The method of claim 9, further comprising secreting the expression product.

11. The method of claim 8, wherein said gene of interest is TFPI.
